(19) United States
(12) Patent Application Publication
(10) Pub. No.: US 2025/0051720 A1
(43) Pub. Date: Feb. 13, 2025

(54) SURFACE COATING FOR SPHEROID GROWTH

(71) Applicants: Aman Russom, Solna (SE); Harisha Ramachandraiah, Solna (SE); Torbjörn Pettersson, Järna (SE)

(72) Inventors: Aman Russom, Solna (SE); Harisha Ramachandraiah, Solna (SE); Torbjörn Pettersson, Järna (SE)

(21) Appl. No.: 18/718,950

(22) PCT Filed: Dec. 15, 2022
(86) PCT No.: PCT/SE2022/051184

§ 371 Date: Jun. 12, 2024

(30) Foreign Application Priority Data
Dec. 17, 2021 (SE) 2151548-1

(51) Int. Cl. C12N 5/00

(52) U.S. Cl. CPC C12N 5/0068; C12N 5/0062; C12N 2513/00; C12N 2533/30; C12N 2533/90

(57) ABSTRACT

The present invention in a first aspect relates to a method for generating three-dimensional multicellular spheroids from at least one eukaryotic cell, comprising seeding at least one cell onto a scaffold comprising at least one bi-layer comprising a first layer and a second layer (106) with alternating charges, said first layer comprising a polyelectrolyte being biocompatible with said cells and said second layer comprising nanocellulose.

### TECHNICAL FIELD OF THE INVENTION

The present invention relates in general to the field of analytical biochemistry and more specifically to the techniques to generate spheroids from eukaryotic cells. The invention provides a surface coating based on Layer-by-Layer technique (LbL) containing nanocellulose used for cell seeding and growth.

### BACKGROUND TO THE INVENTION

Spheroids are a three dimensional (3D) cell aggregation mass, where number of cells form a solid unit together, periphery of each cell is not individually observable, whereas the overall unit usually presents a clear outline. Spheroids are assumed to represent tumor biology more than conventional two-dimensional (2D) cell culture system, as has previously shown that 3D cell aggregates produce a different proliferation areas, different gene expression patterns and cellular features in the spheroid. The cell-cell interaction is an important characteristic of 3D cultures and the oxygenation and nutrient gradient of the cells in spheroids mimic the microenvironment, which is missing in 2D cultures. All these features of 3D culture model are useful for specific research aspects. There is evidence that spheroids can be generated from a single cell type or mixtures of cell types such as tumor, stromal, and immune cells. The cells are self-organized in a complex cell-matrix and cell-cell interaction that imitate functional characteristics of grown tissue. Most importantly, spheroids can be monitored easily for practical daily observations, therefore, spheroids used as a model to identify novel anti-cancer therapeutics for several cancer diseases such as breast, colon and prostate.

Methods of spheroid formation have been progressed overtime. Hanging drop techniques, spheroids formed in specialized plates such as on the bottom of culture plate lids, culture of cells on non-adherent surfaces, spinner flask cultures, and rotary cell culture systems however, these conventional hanging drop spheroid formation methods produce high range of spheroids size. Recently, a pressure-assisted network for droplet accumulation (PANDA) system, has been developed, where multichannel fluidic system and a hanging drop cell culture combined to produce uniform 3D microtissue. This technology has a low-throughput and the technological complexity could limit its use.

Various microfluidic platforms (spheroids on a chip) have also been developed to increase the efficiency of spheroid formation. Cell culture plate using polydimethylsiloxane (PDMS) as a plate constituent has presented as a mass spheroids formation method. These techniques, still suffer from limitations such as long-term culture and device manufacturing complexity and compatibility with drugs. Most importantly, these techniques are often not applicable with existing liquid handling robots for high-throughput screening (HTS).

Tissue models and 3D tumor can also be generated by cells seeding on pre-fabricated scaffolds, or matrices, which designed to resemble the in-vivo extracellular matrix (ECM). Cells attach, migrate, and fill the interstices within the scaffold to form 3D structures. This method has been used for an in-vitro tissue regeneration; multiple forms of the combined polymers are used for spheroid generation, including polystyrene (PS) and polycaprolactone (PCL), poly(vinylidene fluoride) (PVDF) has been used to form substrate for neural stem cell regeneration. Un-modified CNF hydrogel has been shown to be capable to form 3D spheroids and small tissue-cell aggregates of various cell lines.

Scaffolds have also been produced by natural proteins providing ECM such as alginate, collagen, laminin, gelatin, Matrigel and chitosan for spheroid formation and tissue regeneration. The spheroids generation in scaffolds applied by mixing the cells in a liquid state with scaffold materials such as carboxymethyl cellulose and composite hydrogels of TEMPO-oxidized cellulose nanofibrils and nanofibrous chiral Cu(II) aspartate polymer, followed by culturing in a microplate well. For example by using One part is Eu(III) 2-(2-aminobenzamido) benzoic acid complex functionalized carboxymethyl cellulose (CMC) with another part 2, 6-pyridinedicarboxylic acid functionalized CMC gels can be formed that incorporate cells that can grow inside the gel. (Hai et al., 2019) Cells can also be added to pre-prepared microplate. Thereafter, the formed tissue released from scaffold using enzymatic biodegradation, however, the enzymatic degradation may affect in the function of formed spheroids.

The nanofibrillated cellulose (CNF) produced from native cellulose can be used as reinforcement agent in polymer composite materials. It has unique features such as high strength and stiffness, low weight and biodegradability. Cellulose nanofibrils have been widely studied to develop nano-thin layers assembled on the surface of different substrates using the ability of physisorption onto different materials. CNF deposited alternatively with positively charged polymers to form a multifunctional LbL coating that was modified by grafting antibodies to the cellulose on the surface has been used to produce a microfluidic chip for immunoaffinity capture of cancer cells that strongly attach to the surface via antibody-antigen interaction and followed by release via enzymatic digestion of the cellulose surface to destroy the surface to recover the captured cancer cells (Kumar et al., 2020).

Layer by Layer coating onto medical devices is known from e.g. WO2004/025332.

This invention describes surface coating strategy using Layer-by-Layer (LbL) technique, where biocompatible polymers assembles alternatively with nanocellulose on the surface of different material serve as surface for cell growth were the fibrillar like structure of the nanocellulose are vital to form spheroids.

### SUMMARY OF THE INVENTION

The present invention provides a method for generating three-dimensional multicellular spheroids from at least one eukaryotic cell, comprising seeding at least one cell onto a scaffold comprising at least one bi-layer comprising a first layer and a second layer with alternating charges, said first layer comprising a polyelectrolyte being biocompatible with said cells and said second layer comprising nanocellulose, and incubating the seeded cell or cells and the scaffold for generation of three-dimensional multicellular spheroids.

The seeded cell or cells and the scaffold are incubated under conditions suitable for generation of three-dimensional multicellular spheroids.

Such conditions are known in the art, and may be incubation in 5% CO2 at 37° C.

In one embodiment, the nanocellulose is cellulose nanofibrils or cellulose nanocrystals.

In one embodiment, the nanocellulose is cationic nanocellulose, such as cationic cellulose nanofibrils, or cationic cellulose nanocrystals, and the biocompatible polyelectrolyte is a negatively charged polyelectrolyte.

In one embodiment, the negatively charged polyelectrolyte is selected from the group consisting of polyacrylic acid (PAA), poly(styrene sulfonate) (PSS), anionic starch, anionic nanocellulose, pectin, poly(methacrylic acid) (PMMA), anionic poly(acrylamid) (APAM), poly-aspartic acid, poly-glutamic acid, carboxymethyl cellulose, carboxymethyl dextrans, alginates, pectins, gellan, carboxyalkyl chitins, carboxymethyl chitosans, sulfated polysaccharides, and glucoproteins.

In one embodiment, the nanocellulose is anionic nanocellulose, such as anionic cellulose nanofibrils, or anionic cellulose nanocrystals, and the biocompatible polyelectrolyte is a positively charged polyelectrolyte.

In one embodiment, the positively charged polyelectrolyte is selected from the group consisting of polyethyleneimine (PEI), poly(allylamine hydrochloride) (PAH), polydiallyldimethylammonium chloride (polyDADMAC), chitosan, cationic starch, cationic nanocellulose, cationic poly(acrylamid) (CPAM), and aminoalkylated polysaccharides.

In one embodiment, not all bi-layers comprise the same biocompatible polyelectrolyte.

In one embodiment, at least one outermost bi-layer has an outermost layer comprising nanocellulose.

In one embodiment, the scaffold is arranged on a solid support.

In one embodiment, the eukaryotic cell or cells are mammalian cells, such as human cells.

### Definitions & Abbreviations

Terms and abbreviations in the present disclosure shall have the meaning usually ascribed to them in the art unless indicated otherwise. In so far as the present disclosure relates to cellulose nanomaterials the terms shall have the meaning as defined in the standard ISO/TS 20477:2017.

LbL=Layer-by-layer.

The term “spheroid” denotes a three dimensional (3D) cell aggregation mass, where number of cells form a solid unit together, periphery of each cell is not individually observable, whereas the overall unit usually presents a clear outline.

The term cellulose nanofibril (CNF) is a cellulose nanofibre composed of at least one elementary fibril, containing crystalline, paracrystalline and amorphous regions, with aspect ratio usually greater than 10, which may contain longitudinal splits, entanglement between particles, or network-like structures. The dimensions are typically 3-100 nm in cross-section and typically up to 100 μm in length. The aspect ratio refers to the ratio of the longest to the shortest dimensions. (see also ISO/TS 20477:2017).

Cellulose nanocrystal (CNC) is nanocrystal predominantly composed of cellulose with at least one elementary fibril, containing predominantly crystalline and paracrystalline regions, with aspect ratio of usually less than 50 but usually greater than 5, not exhibiting longitudinal splits, inter-particle entanglement, or network-like structures. The dimensions are typically 3-50 nm in cross-section and 100 nm to several μm in length depending on the source of the cellulose nanocrystal. The aspect ratio refers to the ratio of the longest to the shortest dimension. (see also ISO/TS 20477:2017).

The terms nanocellulose (NC) is cellulose nanomaterial composed predominantly of cellulose, with any external dimension in the nanoscale, or a material having internal structure or surface structure in the nanoscale, with the internal structure or surface structure composed predominantly of cellulose. Some cellulose nanomaterials can be composed of chemically modified cellulose. This generic term is inclusive of cellulose nano-object, cellulose nanocrystal and cellulose nanofibril.

### DESCRIPTION OF THE FIGURES

FIG. 1: (A) Schematic illustration of a bi-layer of a cell cultivation scaffold as used in the present invention. (B) Schematic illustration of a cell cultivation scaffold as used in the present invention.

FIG. 2: Spheroid characterization on LbL coated surface compared to poly-HEMA coating (A) spheroids generation from HCT 116 cell line on LbL coated surfaces correlated to different bi-layers number and cell concentration, data presented as mean and standard deviation (SD) of formed spheroids. (B) Spheroids generation from HEK 293T cell line on LbL coated surfaces, data presented as mean and (SD). (C) Spheroids formed on poly-HEMA coating of both cell lines. (D) Fluorescent image of formed spheroid on poly-HEMA surface, Hoechst fluorescence represent nuclear staining, merged image represents all staining. Scale bar (50 μm).

FIG. 3: Spheroids proliferation overtime on LbL coating. (A) Progression of HCT 116 spheroids size overtime the error bar representing the SD between grown spheroids. The bright field images represent HCT 116 proliferated spheroid at 3, 5 and 10 days. (B) HEK 293T spheroids size overtime, bright field images of HEK spheroid growing, scale bar (10 μm).

FIG. 4: Spheroids viability analysis. (A) The checkered bars representing the mean fluorescence intensity of viable cells (calcein stained cells) and the striped bars representing the dead cells (PI stained cells) the error bars presenting the SD variation between the analyzed spheroids (n=4). (B) The image shows the Live/Dead stained spheroid, Hoechst, viable cells (calcein), dead cells (PI stain) and the composite of the stained spheroid, scale bar (50 μm).

FIG. 5. Treatment of HCT 116 spheroids with an anti-cancer drug. The graph representing the mean fluorescent intensity of viable (●) and dead (▪) cells before and after treatment of spheroids with an anti-cancer drug of specific concentrations of the drug, the error bars presenting the SD between the analyzed spheroids (n=4).

### DETAILED DESCRIPTION OF THE INVENTION

The present invention aims to provide improved methods overcoming at least some of the drawbacks and problems of the prior art relating to growing spheroids. The present invention provide a method that enhance the spheroid formation and proliferation from a low cell concentration. In addition, the biocompatibility of LbL coating verified by the evaluation of formed spheroids viability. Moreover, the feasibility of formed spheroids to be used for anti-cancer drug screening has been demonstrated. The results indicate the efficacy of our platform to be used for spheroid formation as a model for anti-cancer drug screening. The developed coating strategy could be applied on different surface materials for biological and pharmaceutical application.

The present invention in a first aspect relates to a method for generating three-dimensional multicellular spheroids from at least one eukaryotic cell, comprising seeding at least one cell onto a scaffold comprising at least one bi-layer comprising a first layer and a second layer (106) with alternating charges, said first layer comprising a polyelectrolyte being biocompatible with said cells and said second layer comprising nanocellulose,

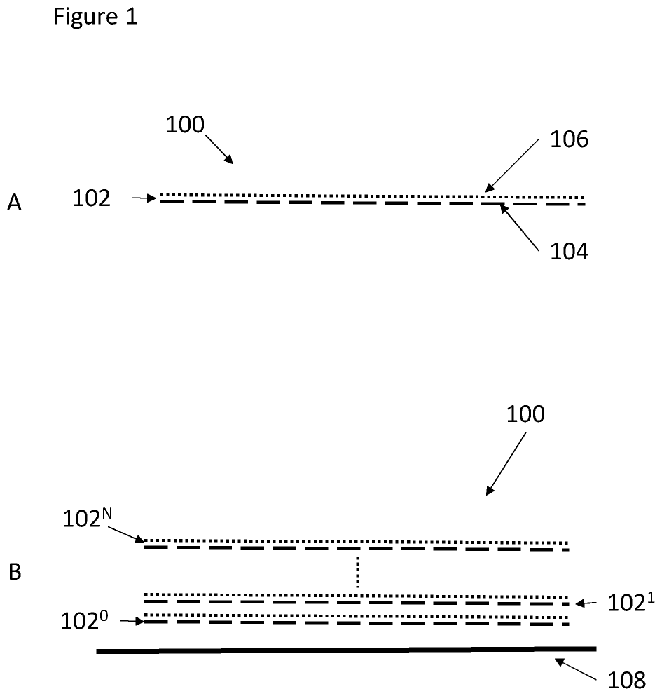

With reference to FIG. 1A, a bi-layer (102) of a scaffold (100) comprise a first layer (104) and a second layer (106) with alternating charges, said first layer (104) comprising a polyelectrolyte being biocompatible with the cells to be cultivated on the scaffold and said second layer (106) comprising nanocellulose. FIG. 1B shows a scaffold (100) comprising a plurality (N) of bi-layers (102^{0-N}) arranged on a solid support (108).

The scaffold used in the invention can be produced by using layer-by-layer (LbL) assembly. This is a technique for building thin coatings of polyelectrolytes or nanocellulose using components of alternating charges between different layers. The polyelectrolyte used can be of natural origin or synthetic origin and can be positively or negatively charged.

Methods for formation of multiple bi-layers with alternating charges for producing the cell cultivation scaffold used in the present invention are known in the art. Generally, a precursor layer is formed by covering the surface with cationic or anionic polyelectrolyte solution. The substrate is incubated to facilitate adsorption of the polyelectrolyte to the surface. The polyelectrolyte solution is removed and the substrate is washed with water or saline solution. Followed by adding of anionic polyelectrolyte solution and incubated for adsorption followed by washed with water or saline solution, in this way one bi-layer have been formed. This can be followed multiple times (up to hundreds of times or more) by sequentially adsorbing additional layers of cationic polyelectrolyte, rinsing, adsorbing additional layers of anionic polyelectrolyte, rinsing, repeating and ending when a desired number of layers is obtained. Preferably, the outermost layer is nanocellulose.

The adsorption steps can be done by solution casting, spray coating or spin coating.

The first, and if applicable also the second, nanocellulose used may be cellulose nanofibrils or cellulose nanocrystals charged by either carboxymethylation, TEMPO oxidation, sulphonation or amination.

Examples of positively charged polyelectrolytes useful in the present invention are polyethyleneimine (PEI), poly(allylamine hydrochloride) (PAH), polydiallyldimethylammonium chloride (polyDADMAC), chitosan, cationic starch, cationic nanocellulose, cationic poly(acrylamid) (CPAM), and aminoalkylated polysaccharides.

Examples of negatively charged polyelectrolytes useful in the present invention are polyacrylic acid (PAA), poly(styrene sulfonate) (PSS), anionic starch, anionic nanocellulose, pectin, poly(methacrylic acid) (PMMA), anionic poly(acrylamid) (APAM), poly-aspartic acid, poly-glutamic acid, carboxymethyl cellulose, carboxymethyl dextrans, alginates, pectins, gellan, carboxyalkyl chitins, carboxymethyl chitosans, sulfated polysaccharides, and glucoproteins.

The technology enable cell seeding down to single cell for monoclonal spheroid formation.

The non-adhesive properties of modified nanocellulose when built up as a scaffold on the solid support, leads to inhibition of cell attachment to the surface, that enhances the spontaneous cells gathering and promotes cell-cell adhesive molecules to form spheroids. Furthermore, a negative charge of the nanocellulose outermost layer of the scaffold can serve as a repulsive force due to the negative charge of the cell membrane that may promote cell collection and adhesion by integrin proteins on the cell surface.

It was observed that there was no significant difference in the number of formed spheroids in the wells coated with different number of bi-layers, indicating no significant correlation between the number of bi-layers and the number of formed spheroids. However, there was variation in the spheroid formation rate between the two cell lines. The cell lines chosen in this study are native to the human species and have shown significant representation of the cancer cell metabolism and proliferation. It has been noticed these cell lines can possibly hold different compatibility and sensitivity toward the culture surface, where the cell proliferation rate and spheroid formation rate can vary from each cell line under certain circumstances.

The correlation between the cell seeding concentration and the number of formed spheroids on the coated surface was studied. The rate of spheroid formation is positively correlated to the cell concentration, indicating that a higher seeding concentration can be beneficial in term of generating a relatively larger number of spheroids, within the same culture condition.

The previous studies have shown that on 3D tumor spheroid culture, the seeding concentration tends to be significantly higher in the microwell plate. In LbL coated wells spheroids were generated at low seeding cell concentration in a standard 96 well plate. It was also observed that on poly-HEMA coated wells under the same culture condition; tumor spheroid cannot form at low seeding concentration.

Consequently, the method according to the invention can be used to form spheroids with high-throughput and applying simple culture conditions.

Furthermore, spheroid proliferation can be achieved. Interestingly, the spheroids proliferated on LbL coating without compromising its viability, indicating the biocompatibility of the spheroid generation platform disclosed herein.

Moreover, the formed spheroids on the LbL coating system show positive response to an anti-cancer drug, demonstrating the ability of the LbL platform to generate functional spheroids can be used simply for biological and pharmaceutical applications.

In summary, altogether, these data show the capability of our coating strategy to generate spheroids, without compromising the viability. The feasibility of the generated spheroids on the LbL surface to be used as a model to test the anti-cancer drug has been established (Example 3).

The examples below are provided to enable the skilled person to understand the present disclosure and is not intended to limit the scope of the invention, which is defined by the appended claims. All references cited herein are expressly incorporated in their entirety by reference.

### EXAMPLES

### Material

Cell lines HCT 116 and HEK 293T were purchased from American Tissue Type Collection. Dulbecco's modified Eagle's medium (DMEM), McCoy's 5A medium, fetal bovine serum (FBS), non-essential amino acids and Penicillin-Streptomycin were purchased from Sigma Aldrich, Germany. Trypan Blue solution 0.4% liquid sterile-filtered and Trypsin EDTA solution 1× (Sigma Aldrich, Germany). Polyallylamine hydrochloride (PAH) from Polyscience, Polyethylenimine (PEI) from Sigma Aldrich, carboxymethylated cellulose nanofibrils (CNF) with a charge density of 0.6 meq g⁻¹ from RISE, Sweden. Nuclear fluorescent stain (Hoechst), Calcein-AM were purchased from (Sigma Aldrich), and Propidium Iodide (PI) (Thermofisher scientific). Irinotecan hydrochloride (Sigma Aldrich). Poly(2-hydroxyethyl methacrylate) Poly-HEMA (Sigma Aldrich, Germany). TC-plate 96 well Suspension F (SARSTEDT AG & amp; Co.) 96 Well Cell Polystyrene Sterile, 6.4 mm diameter size, and 360 μL total volume.

### Example 1: Layer by Layer Coating of a Cell Cultivation Device

Nanometer thin films of CNF were built on the inner surfaces of the 96 standard polystyrene well plates (SARSTEDT AG &Co.) using LbL assembly. Initially, a precursor layer was formed by covering the bottom of the wells with cationic solution of (50 mg/L) PAH prepared in (10 mmol) NaCl, pH 7.5, incubated for 10 min for adsorption at room temperature (RT), then the solution was removed by suction and washed with (200 μl) of Milliq water (MQ water), pH 6.9 for 3 times. This was followed by adding anionic CNF solution (50 mg/L) prepared in MQ H₂O according to protocols as previously described (Erlandsson et al., 2018) into the wells and incubated for 10 minutes at RT. Then the CNF solution was removed by suction and the wells were washed with (200 μl) of MQ water, 3 times and (50 mg/L) PEI solution prepared in (10 mmol) NaCl, pH 7 was added into the wells and allowed 10 min to adsorb, resulting in PAH (CNF/PEI) bi-layers. The process with PEI and CNF was repeated 2, 3, 4 and 5 times in separated wells to have wells coated with 2, 3, 4 and 5 bi-layers, and ending when the outermost layer in all coated wells was CNF.

The LbL assembly layers were characterized in terms of outer cellulose surface uniformity of assembled layers, using Atomic force microscopy (AFM) (data not shown). Control wells were prepared for the LbL coating, non-coated wells and wells treated only with the CNF solution which was not proceeded by PAH initial layer followed by washing with MQ water. In addition, completely dried LbL coated wells (5 bi-layers) and re-wetted 5 bi-layers coated wells were also used as controls.

### Example 2: Spheroids Generation

### Initial Culture

Two cancer cell lines were used for the spheroid generation experiments. Dulbecco's modified Eagle's medium was used for HEK 293T culture and McCoy's medium was used for HCT 116 culture, both media were supplemented with 10% FBS, 100 U/mLPenicillin and 1 g 10 ml−1 streptomycin. Cells incubated in 5% CO2 at 37° C. Cells were harvested every 2-3 days by using 0.05% trypsin and incubating for 5 min at 37° C., and then cells were washed with the corresponding media and re-cultured.

### Spheroids Generation in 96 Well Plates

The confluency growing monolayer cultured cells were trypsinized into a single cell suspension with 98-100% viability measured by trypan blue, and counted using a Bio-RAD TC20 Automated Cell counter. The cell suspension of both cell lines were loaded into the coated wells 2, 3, 4 and 5 bi-layers and to the control wells (cellulose coated and dried and re-wetted LbL coated wells) at densities ranging from (3 to 700) cells per ml, the total medium volume in each well was (300 μl) for both cell lines. Non-coated wells for both cell lines were used as controls. The well plates were incubated in the incubator 5% CO₂ at 37° C. The medium was exchanged every 24 hours of culture; the spheroid formation was monitored using optical microscopy, (ZOE Fluorescent cell imager Bio-RAD). To examine the spheroid growth over time, the growing spheroids for each cell seeding concentration were imaged every day for 10 days. The spheroids diameter was measured by ImageJ software.

### Spheroids Formation in Poly-HEMA Coated Well Plate:

Poly-HEMA coated 96 standard well plates were prepared to be used as a comparison coating technique of LbL-CNF coating for spheroid formation. Poly-HEMA (120 mg ml⁻¹) stock solution was prepared in 95% ethanol. The stock solution was incubated with continues mixing at RT overnight. To make a working solution of Poly-HEMA, pipette (1 mL) of Poly-HEMA stock solution into (23 mL) of 95% ethanol to obtain a final concentration of (5 mg/mL), (60 μL) of the prepared solution was added into each well of a 96-well plate and evaporate with lids on at RT inside a sterile hood for 72 hours. The cell suspension of both cell lines were loaded into the poly-HEMA coated wells with the same range of concentration used for LbL-CNF coated wells, however two higher concentrations were used (5000 and 1000). The well plates were incubated in 5% CO₂ at 37° C.

### Assessment of Generated Spheroids Viability:

Viability of formed spheroids was evaluated using a Live/Dead staining kit. Briefly the formed spheroids in the wells were washed with the culture media with gentle pipetting. Spheroids suspended in (200 μl) of culture medium, (0.5 L) of Calcein AM (520 Da) (1 mg/mL), an indicator for live cells, and (5 μL) of propidium iodide (PI (668 Da) (1 mg/mL), an indicator for dead cells. Wells were incubated in dark at 37° C. for 30 min. Hoechst stain was added to the wells and incubated for 10 min. Stained cells were then visualized using confocal laser scanning microscopy (Zeiss, Germany), and images were analyzed using ImageJ software. Spheroids formed on poly-HEMA coated wells were stained with the same method as performed for LbL-CNF coated wells spheroids.

### Results

It was found that that the spheroid formation was established after 24 hrs of incubation on the surface of 2, 3, 4 and 5 bi-layers coated wells. Cells in the non-coated reference wells and the wells treated with only CNF were not forming spheroids; the seeded cells were grown as monolayer.

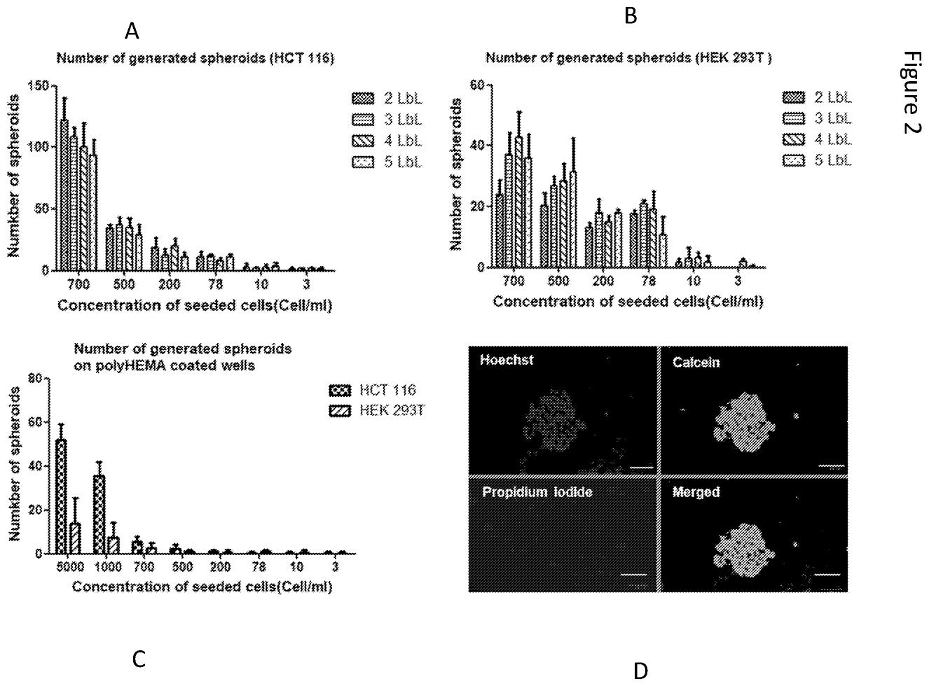

Various parameters of LbL coating were considered to assess and evaluate the potential effect on the spheroid's formation. We have evaluated the spheroid formation with a variety of cell concentration (3-700) cells/mL on the LbL coated wells with different number of bi-layers. We observed that the spheroids formation rate is higher with the high concentration of seeded cells for both cell lines. While, there was no significant difference in spheroids formation correlated to multiple bi-layers number. However, there is a possible deviation on its enhancing effect for spheroids formation when applied with different cell lines. The average number of formed spheroids for HCT 116 and HEK 293T cell lines at higher concentration was (122±17) and (42±8) respectively, and at lower concentration (1±2) and (2±1) respectively. The dried and re-wetted LbL control wells had low capacity to form spheroids with high background of single cells in the well, the average number of spheroids was (5±3), and no formed spheroids at low cell concentration. In contrast, in poly-HEMA coated wells spheroids were formed within 48 hrs at higher concentration, (5000, 1000 and 700) cells/mL (52±6), (35±6) and (5±2) respectively for HCT 116, and (14±11), (8±6) and (3±2) respectively for HEK 293T (FIG. 2A-C).

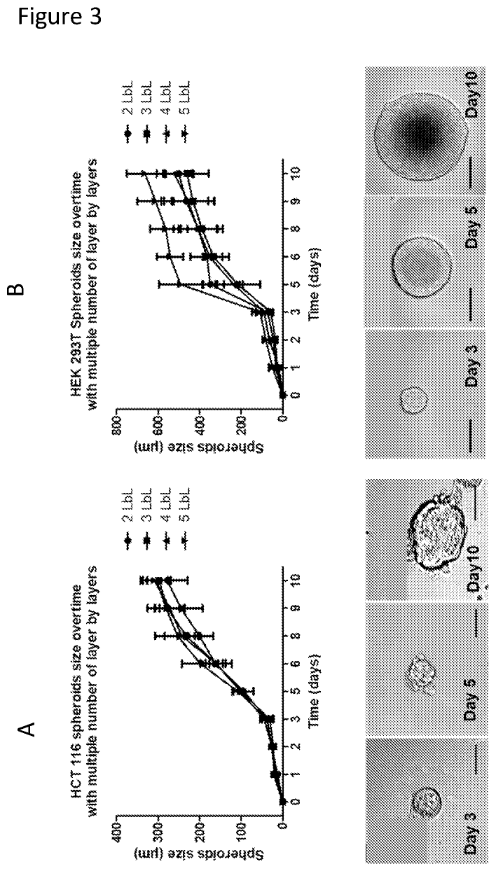

Furthermore, systematic analysis has been conducted, and with longer observation duration for the generated spheroids. A generalized positive correlation can be more accurately concluded between the spheroid's diameter (size) and the culture time for 10 days. However, the average spheroid size (n=5) of HCT 116 and HEK 293T cell lines on the 5 bi-layers coated wells was (309, 663) μm respectively, and (277, 500) μm respectively on the 2 bi-layers coated wells as shown in FIG. 3.A and B. Nevertheless, the difference of spheroids size was statistically not significant in correlation to multiple bi-layers number. Spheroids formed on poly-HEMA coated wells were slightly proliferated (103) μm (data not shown).

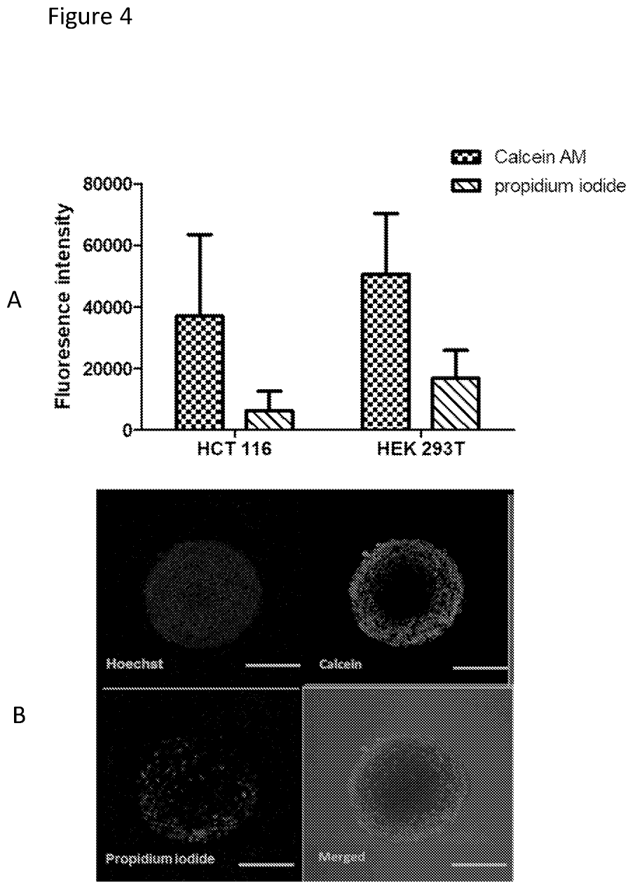

The viability of LbL coating formed spheroids was measured, along with poly-HEMA spheroids of both cell lines. The fluorescence intensity was analyzed for stained viable and apoptotic cells (n=4). As seen in FIG. 4.A and B. the apoptosis of LbL growing spheroids (PI-positive cells) was started after 10 days of cell culture. Although, the mean fluorescence intensity of viable cells (calcein AM-positive cells) was higher than dead cells (PI-positive cells) after 10 days culture for both cell lines.

### Example 3 Anti-Cancer Drug Treatment of HCT 116 Formed Spheroids

The formed spheroids from HCT 116 cell line were treated with anti-cancer drug to investigate the functionality of formed spheroids for drug screening. Irinotecan hydrochloride (5 mg/mL) drug was used to test the effect of anti-cancer drug in the colon cancer cell line (HCT 116) generated spheroids. The spheroids were suspended in the wells with the medium; and treated with different concentration of the drug (100, 50 and 10 μg ml⁻¹), non-treated spheroids were used as a control. All the wells were incubated for 24 hrs, in the incubator 5% CO₂ at 37° C. Then wells were washed with the media and stained with Live/Dead stain as mentioned earlier, followed by imaging using confocal laser scanning microscopy and the images analyzed with ImageJ software.

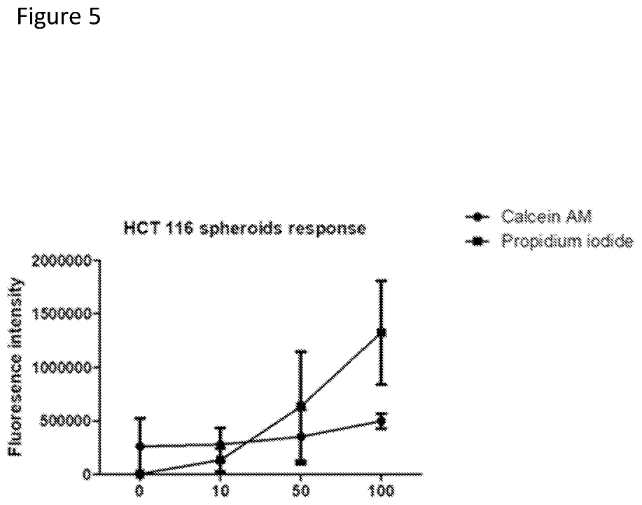

The response of the spheroids estimated by the fluorescence intensity of viable and dead cells in each spheroid (n=4). We found that the spheroids treated with anti-cancer drug had lower viability compared to the non-treated spheroids. The highest response was at high concentration of the drug (100 μg/mL) as seen in FIG. 5.

### REFERENCES

Erlandsson et al. (2018). On the mechanism behind freezing-induced chemical crosslinking in ice-templated cellulose nanofibril aerogels. Journal of Materials Chemistry, 6, 19371-19380.
Kumar et al. (2020). Multi-layer assembly of cellulose nanofibrils in a microfluidic device for the selective capture and release of viable tumor cells from whole blood. Nanoscale, 12, 21788.
Hai et al. (2019). Anions reversibly responsive luminescent nanocellulose hydrogels for cancer spheroids culture and release. Biomaterials, 194, 161-170.

1-10. (canceled)

11. A scaffold for generating three-dimensional multicellular spheroids from at least one eukaryotic cell, the scaffold comprising: a first layer and a second layer with alternating charges, said first layer comprising a biocompatible polyelectrolyte and said second layer comprising nanocellulose, wherein the polyelectrolyte is a positively charged polyelectrolyte or a negatively charged polyelectrolyte.

12. The scaffold according to claim 11, wherein the polyelectrolyte is of natural origin or synthetic origin.

13. The scaffold according to claim 11, wherein the nanocellulose is selected from the group consisting of cellulose nanofibrils or cellulose nanocrystals.

14. The scaffold according to claim 13, wherein the cellulose nanofibrils or cellulose nanocrystals are charged by carboxymethylation, TEMPO oxidation, sulphonation or amination.

15. The scaffold according to claim 11, wherein the nanocellulose is cationic nanocellulose or cationic cellulose nanocrystals, and the biocompatible polyelectrolyte is a negatively charged polyelectrolyte.

16. The scaffold according to claim 11, wherein the nanocellulose is anionic nanocellulose or anionic cellulose nanocrystals, and the biocompatible polyelectrolyte is a positively charged polyelectrolyte.

17. The scaffold according to claim 11, wherein the positively charged polyelectrolyte is selected from the group consisting of polyethyleneimine (PEI), poly(allylamine hydrochloride) (PAH), polydiallyldimethylammonium chloride (polyDADMAC), chitosan, cationic starch, cationic nanocellulose, cationic poly(acrylamid) (CPAM), and aminoalkylated polysaccharides.

18. The scaffold according to claim 11, wherein the negatively charged polyelectrolyte is selected from the group consisting of polyacrylic acid (PAA), poly(styrene sulfonate) (PSS), anionic starch, anionic nanocellulose, pectin, poly(methacrylic acid) (PMMA), anionic poly(acrylamid) (APAM), poly-aspartic acid, poly-glutamic acid, carboxymethyl cellulose, carboxymethyl dextrans, alginates, pectins, gellan, carboxyalkyl chitins, carboxymethyl chitosans, sulfated polysaccharides, and glucoproteins.

19. The scaffold according to claim 11, wherein not all bi-layers comprise the same biocompatible polyelectrolyte.

20. The scaffold according to 11, wherein at least one outermost bi-layer has an outermost layer comprising nanocellulose.

21. The scaffold according to claim 11, wherein the scaffold is arranged on a solid support.

22. A method for generating three-dimensional multicellular spheroids from at least one eukaryotic cell, using the scaffold of claim 11, the method comprising: seeding at least one cell onto the scaffold, and incubating the seeded cell or cells and the scaffold for generation of three-dimensional multicellular spheroids.

23. The method according to claim 22, wherein the eukaryotic cell or cells are mammalian cells.

24. A method of producing a scaffold according to claim 11, wherein the method comprises the steps of:
a. providing a negatively charged polyelectrolyte solution selected from the group consisting of polyacrylic acid (PAA), poly(styrene sulfonate) (PSS), anionic starch, anionic nanocellulose, pectin, poly(methacrylic acid) (PMMA), anionic poly(acrylamid) (APAM), poly-aspartic acid, poly-glutamic acid, carboxymethyl cellulose, carboxymethyl dextrans, alginates, pectins, gellan, carboxyalkyl chitins, carboxymethyl chitosans, sulfated polysaccharides, and glucoproteins,
b. providing a positively charged polyelectrolyte solution selected from the group consisting of polyethyleneimine (PEI), poly(allylamine hydrochloride) (PAH), polydiallyldimethylammonium chloride (polyDADMAC), chitosan, cationic starch, cationic nanocellulose, cationic poly(acrylamid) (CPAM), and aminoalkylated polysaccharides;
c. providing a surface material;
d. formation of a precursor layer by covering a surface with a first polyelectrolyte solution with cationic or anionic charge;
e. incubating the first polyelectrolyte solution on said surface to facilitate adsorption of the first polyelectrolyte to the surface;
f. removing of the non-absorbed first polyelectrolyte solution and washing of the surface with water or saline solution;
g. adding of a second polyelectrolyte solution comprising the opposite charge of the first polyelectrolyte solution;
h. incubating the second polyelectrolyte solution to facilitate adsorption of the second polyelectrolyte solution to the surface with the absorbed first polyelectrolyte solution; and
i. removing of the second polyelectrolyte solution from the surface with the absorbed first and second polyelectrolyte solution and washing with water or saline solution.

25. The method of producing a scaffold according to claim 24, wherein the absorption in step e. and h. are performed by solution casting, spray coating or spin coating.

26. The method of producing a scaffold according to claim 24, wherein the method comprises repeating step d. to i. at least once.

27. The method of producing a scaffold according to claim 24, wherein the method further comprises a step j., said step comprising adding an outermost layer comprising nanocellulose.
